(19) United States
(12) Patent Application Publication
(10) Pub. No.: US 2025/0356980 A1
(43) Pub. Date: Nov. 20, 2025

(54) Methods, Systems, and Apparatus for Tapering or Uptitrating Drug Dosages

(71) Applicant: The United States Government as represented by the Department of Veterans Affairs, Washington, DC (US)

(72) Inventors: Constance Fung, Sherman Oaks, CA (US); Lara Kierlin, Bellevue, WA (US)

(21) Appl. No.: 19/284,452

(22) Filed: Jul. 29, 2025

Related U.S. Application Data
(63) Continuation of application No. 17/773,823, filed on May 2, 2022.
(60) Provisional application No. 62/928,896, filed on Oct. 31, 2019.

(51) Int. Cl. G16H 20/10; A61J 7/00

(52) U.S. Cl. CPC G16H 20/10; A61J 7/0007

(57) ABSTRACT

This disclosure relates to a method tapering or uptitrating a drug, e.g., benzodiazepines or non-benzodiazepine sedative hypnotics. Also disclosed is an apparatus for cutting or splitting a pill which can be used to blind or mask a dosage during a taper or uptitration plan. Also disclosed are methods of using the apparatus.

### CROSS-REFERENCE TO RELATED APPLICATIONS

This application is a continuation of U.S. application Ser. No. 17/773,823, filed May 2, 2022, which is a national stage filing under 35 U.S.C. § 371 of International Application No. PCT/US2020/058544, filed Nov. 2, 2020, which claims the benefit of U.S. Provisional Application No. 62/928,896, filed Oct. 31, 2019, the entireties of each of which are incorporated herein by reference.

### STATEMENT OF GOVERNMENT SUPPORT

This invention was made with federal government support under grant number AG057929, awarded by the National Institutes of Health. The government has certain rights in the invention.

### FIELD OF THE INVENTION

The present invention is directed to treating a subject with drugs, such as pharmaceuticals, including methods and systems thereof, more particularly, tapering and uptitrating doses of the drugs in a way that utilizes psychological phenomenon associated with taking pills to increase the success of a taper or uptitration. The present invention is also directed to an apparatus and method to produce drug dosages in specific dose levels, suitable for blinded or masked administration to a subject.

### BACKGROUND

Some drugs such as sedative-hypnotics, anxiolytics, and pain medications may be challenging to discontinue. Successful discontinuation can be enhanced by slowly reducing the drug (i.e., tapering). Yet a slow, gradual taper can be insufficient to achieve successful discontinuation, even in the absence of physiological withdrawal symptoms. The expectancy of the drug's effects (placebo effects) and of withdrawal symptoms (nocebo effects) can also impair an individual's chances of discontinuing the drug. Accordingly, a need exists for new systems and methods designed to improve drug tapering. These needs and others are met by the following disclosure.

### SUMMARY

In one aspect, this disclosure relates to a method of tapering or uptitrating a drug comprising: receiving, at a computing device, a patient-derived parameter comprising at least one of: a Likert-like scale response indicative of the patient's motivation to begin or continue a drug tapering or uptitrating plan, a Likert-like scale response indicative of the patient's subjective perception of a drug tapering or uptitrating plan, the patient's blood pressure, the patient's heart rate, a Likert-like scale response indicative of the patient's subjective report of global well-being, or Clinical Institute Withdrawal Assessment Scale (CIWA-B) scores for benzodiazepines or non-benzodiazepine sedative hypnotics; receiving, at a computing device, a provider-derived parameter comprising at least one of: a baseline dose, a desired end dose, a number of days per week the drug is taken, a day of the week the drug is taken, a number of weeks for tapering, a number of digits following a decimal place for the drug dose, a day for being drug-free days, a function of the taper plan, a strength of the taper plan, a specific dose strength on certain days, an upper bound for percentage reduction during each week or partial week, a lower bound for percentage reduction during each week or partial week, or a maximal percent reduction per week; generating, by the computing device, based on the at least one patient-derived parameter, the at least one provider-derived parameter, and the drug, a taper or uptitrating plan associated with the drug and the patient, wherein the taper or uptitrating plan is indicative of a plurality of dosage amounts of the drug for each of a plurality of specific days; determining, by the computing device, based on the taper or uptitrating plan, a first dosage amount of the plurality of dosage amounts of the drug; and sending, by the computing device, an indication of the first dosage amount.

In a further aspect, disclosed is a method of tapering or uptitrating a drug comprising: receiving, at a computing device, at least one parameter associated with a drug and a patient identifier; generating, by the computing device, based on the at least one parameter and the drug, a taper plan associated with the drug and the patient identifier, wherein the taper plan is indicative of a plurality of dosage amounts of the drug for each of a plurality of specific days; determining, by the computing device, based on the taper plan, a first dosage amount of the plurality of dosage amounts of the drug; and sending, by the computing device, an indication of the first dosage amount.

In a still further aspect, disclosed is an apparatus comprising: a body having a central axis, wherein the body defines a plurality of receptacles that are spaced around the central axis, wherein the plurality of receptacles define a perimeter; a pill cutter disposed on the perimeter defined by the plurality of receptacles, the pill cutter comprising: a platform that is configured to receive a pill; a cutting element that is coupled to the platform and configured to move toward and away from the platform in order to cut the pill received on the platform; and an actuator that is configured to cause the blade to move toward and away from the platform.

Still other objects and advantages of the present disclosure will become readily apparent by those skilled in the art from the following detailed description, which is shown and described by reference to preferred aspects, simply by way of illustration of the best mode. As will be realized, the disclosure is capable of other and different aspects, and its several details are capable of modifications in various respects, without departing from the disclosure. Accordingly, the description is to be regarded as illustrative in nature and not as restrictive.

### BRIEF DESCRIPTION OF THE DRAWINGS

The accompanying drawings, which are incorporated in and constitute a part of this specification and together with the description, serve to explain the principles of the disclosure.

FIG. 1. is a flow-chart illustrating the interrelatedness of psychological and physiological effects related to drug withdrawal. See Stewart-Williams, “The Placebo Puzzle: Putting Together the Pieces,” Health Psychology Vol. 24, No. 2, 198-206 (2004), which is hereby incorporated by reference in its entirety for its teaching of the theories of the placebo effect.

FIG. 2 is a flow-chart illustrating an exemplary method for generating and optionally modifying a drug tapering plan.

FIG. 3 illustrates an exemplary embodiment of a pill cutting and filling apparatus.

FIG. 4 illustrates an exemplary embodiment of a pill cutting and filling apparatus in which the pill cutter can be removably detached from a central bore of the lower body portion.

### DETAILED DESCRIPTION

The present invention can be understood more readily by reference to the following detailed description of the invention and the Examples included therein.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

### A. Definitions

Listed below are definitions of various terms. These definitions apply to the terms as they are used throughout this specification, unless otherwise limited in specific instances, either individually or as part of a larger group.

As used in the specification and in the claims, the term “comprising” can include the aspects “consisting of” and “consisting essentially of.”

As used in the specification and claims, the singular forms “a,” “an,” and “the” include plural referents unless the context clearly dictates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

“Optional” or “optionally” means that the subsequently described event or circumstance may or may not occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; anti-cancer and anti-neoplastic agents such as kinase inhibitors, poly ADP ribose polymerase (PARP) inhibitors and other DNA damage response modifiers, epigenetic agents such as bromodomain and extra-terminal (BET) inhibitors, histone deacetylase (HDAc) inhibitors, iron chelators and other ribonucleotides reductase inhibitors, proteasome inhibitors and Nedd8-activating enzyme (NAE) inhibitors, mammalian target of rapamycin (mTOR) inhibitors, traditional cytotoxic agents such as paclitaxel, doxorubicin, irinotecan, and platinum compounds, immune checkpoint blockade agents such as cytotoxic T lymphocyte antigen-4 (CTLA-4) monoclonal antibody (mAB), programmed cell death protein 1 (PD-1)/programmed cell death-ligand 1 (PD-L1) mAB, cluster of differentiation 47 (CD47) mAB, toll-like receptor (TLR) agonists and other immune modifiers, cell therapeutics such as chimeric antigen receptor T-cell (CAR-T)/chimeric antigen receptor natural killer (CAR-NK) cells, and proteins such as interferons (IFNs), interleukins (ILs), and mAbs; anti-ALS agents such as entry inhibitors, fusion inhibitors, non-nucleoside reverse transcriptase inhibitors (NNRTIs), nucleoside reverse transcriptase inhibitors (NRTIs), nucleotide reverse transcriptase inhibitors, NCP7 inhibitors, protease inhibitors, and integrase inhibitors; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrhythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, MA), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

“Likert-like scale,” as used herein, refers to a bipolar scaling method, measuring either a positive or negative to a statement. The term refers to a scale with any number of suitable points, e.g., strongly agree, agree, neutral, disagree, strongly disagree, and the like.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

While aspects of this disclosure can be described and claimed in a particular statutory class, such as the system statutory class, this is for convenience only and one of skill in the art will understand that each aspect of this disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or description that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### B. Methods for Tapering Drug Dosages

Placebo and nocebo effects in a subject can occur through the repeated pairing of the active drug in the pill (unconditioned stimulus) with the routine of taking a pill from the pill bottle (conditioned stimulus) as well as through cognitive expectancies, such as the expectancy that the drug will produce a clinical effect. Disrupting this pairing and reducing cognitive expectancies can reduce placebo and nocebo effects in a subject in accordance with many embodiments of the invention. Blinding or masking the daily dosage can challenge cognitive expectancies for the drug effect and is a method for increasing the successful tapering and discontinuation of drugs

Chronic drug use, which may be defined as use of a certain drug for a certain length of time. Chronic use is common in many pharmaceutical drugs, such as hypnotics (e.g., benzodiazepines, such as temazepam) and/or non-benzodiazepine receptor agonists (e.g., “z-drugs,” such as zolpidem), for which chronic use is defined as use for three or more consecutive months in the context of current or prior insomnia symptoms. Chronic hypnotic use is common in the United states, and nearly one in ten older adults is prescribed a benzodiazepine over the course of a year. Almost half of benzodiazepine prescriptions among older adults are for treatment of insomnia. Long-term benzodiazepine use increases with age (0.4% in 18-35 years vs. 2.7% in 65-80 years). Although z-drugs such as zolpidem are sometimes considered as alternatives to benzodiazepines, they are also commonly-prescribed and are also associated with poor balance and worsened memory and psychomotor performance.

In the case of hypnotics, benzodiazepines and z-drugs primarily act upon the γ-aminobutyric acid (GABA) complex in the brain. In older adults with insomnia, they increase total sleep time by 25.2 minutes, decrease nighttime awakenings by 0.63, and improve sleep quality (effect size 0.14). However, the observed effects may not be solely due to the GABA-related effects of these medications.

A growing number of studies suggest that improvements in sleep are also due to a second mechanism: nonspecific, placebo effects. Placebos increase patient-reported total sleep time by 13.5 minutes and decrease sleep latency by 13 minutes. They also have a small to moderate effect on objectively-measured outcomes such as total sleep time, wake after sleep onset, and sleep quality, with a recent meta-analysis concluding that 63% of the therapeutic response to hypnotic medications is achieved in the placebo arm of trials. Placebo effects have also been observed in “active” therapy. A trial that tested the effects of a regimen comprised of 50% active and 50% placebo z-drug found that a 50-50 regimen-maintained treatment response over time, as did the 100% active regimen that was tested in another arm of the trial. Although objective, physiological findings have been documented in patients with insomnia and other conditions (e.g., neural networks in pain and Parkinson's disease), the clinical relevancy of placebo effects is still debated. A recent review of non-hypnotic-related, randomized placebo trials found that placebo interventions did not have important clinical effects, although the authors noted that placebo interventions may influence patient-reported outcomes, which are relevant to insomnia studies since insomnia disorder is diagnosed based upon patient-reported symptoms. Explanations for observed changes in the placebo arm of trials include changes in cognitive expectancy, cognitive dissonance, optimism, regression to the mean, the Hawthorne effect, and social desirability. While these effects are often considered “problematic” in clinical trials, they are potentially important novel targets for cognitive therapy interventions.

Discontinuation of hypnotics is associated with improvements in cognition (visuospatial, concentration, problem solving) and balance, including a 20-30% reduction in postural sway. Discontinuation can be achieved without significant withdrawal symptoms and hypnotic discontinuation may be beneficial to patients, even if they do not manifest behavioral, physiological, cognitive, social, or legal problems associated with hypnotic use and even if they do not meet clinical diagnostic criteria for benzodiazepine abuse, dependence, or misuse.

Chronic insomnia disorder (a condition that may be an independent risk factor for adverse events such as falls) can be effectively treated with cognitive behavioral therapy for insomnia (CBTI), which typically includes stimulus control, sleep restriction therapy, cognitive therapy, relaxation training, and sleep hygiene education. Many patients who use hypnotics for chronic insomnia have not been offered CBTI, even though it is considered first-line therapy. CBTI is more effective long-term than hypnotics in older adults, has few known side effects, and is preferred by patients. Access to CBTI has been poor, but efforts are underway to expand access, by training more providers in CBTI and using alternative delivery models.

Finally, hypnotic discontinuation is part of a broader effort to deprescribe medications that have lost their advantage in the risk-benefit trade-off. Deprescribing initiatives, which focus on prevention of adverse outcomes resulting from hypnotics, are consistent with the National Institute on Aging's mission statement—“to extend the healthy, active years of life.” Due to the adverse health outcomes associated with hypnotic use in older adults, medical professional societies advise discontinuing hypnotics in older patients.

Once individuals become chronic hypnotic users, hypnotic tapering is necessary, because abrupt discontinuation of benzodiazepines may result in serious events such as seizures. Withdrawal symptoms can be minimized or averted with a gradual dose taper. Non-pharmacological treatment of chronic insomnia disorder may be needed, because hypnotic discontinuation may uncover insomnia, and strategies to address attributions of negative symptoms to hypnotic withdrawal (rather than underlying insomnia) may prevent relapse.

Multiple studies have examined the efficacy of interventions for discontinuing hypnotic use. These interventions have either focused on reducing the prescribing of hypnotics (e.g., giving feedback to providers about their prescribing patterns) or on withdrawing the hypnotic. Hypnotic withdrawal strategies range from withdrawal alone (e.g., supervised taper) to withdrawal plus pharmacological interventions (e.g., replace the hypnotic with antidepressants, melatonin) or psychosocial interventions such as CBTI, motivational interviewing, brief psychoeducational interventions, or psychodynamic/psychoanalytic/expressive interventions.

Currently, no hypnotic withdrawal strategies specifically target placebo effects associated with hypnotic use or of any commercially-available medications to promote hypnotic discontinuation. A recent meta-analysis of 10 studies conducted in older adult populations found that supervised withdrawal plus a psychological intervention (e.g., cognitive behavioral therapy) increases the odds of benzodiazepine discontinuation (odds ratio=5.06, 95% CI 2.68-9.57), whereas prescribing interventions modestly increase the odds of benzodiazepine discontinuation (OR=1.43, 95% CI 1.02-2.02) compared to usual care, education placebo, withdrawal with or without drug placebo, or psychotherapy alone. This suggests that new interventions aimed at improving discontinuation of hypnotics should include supervised withdrawal and a psychosocial intervention such as cognitive behavioral therapy. However, supervised withdrawal plus cognitive behavioral therapy may not be sufficient to sustain non-use. Meta-analyses and systematic reviews find that gradual withdrawal regimens (i.e., taper) plus cognitive behavioral therapy (e.g., CBTI), together, are efficacious short-term (up to 3 months post-treatment; RR 1.51, 95% CI 1.15 to 1.98); however, the effects of taper plus cognitive behavioral therapy are not statistically significant at >6 months follow up) compared to taper only (taper versus usual care results in 40% discontinuation at 6 months).

Supervised gradual withdrawal plus a psychosocial intervention such as CBTI is currently the most efficacious method available for achieving short-term (e.g., up to 3 months) hypnotic discontinuation, but additional strategies are needed to increase sustained non-use (e.g., >6 months). The placebo effect is an unexplored target for improving discontinuation rates and is utilized in a number of aspects of the invention to improve discontinuation rates.

Without wishing to be bound by theory, it is generally believed that cognitive expectancies lead to changes in emotion and behavior, as well as schematic processing, which alters attentional focus, all resulting in placebo effects. A number of aspects of the invention utilize an integrative model for a placebo effect that posits that cognitive expectancies are shaped by conditioning procedures and verbal persuasion, as well as observational learning. These aspects integrate the model where conditioning occurs through the repeated pairing of the active drug in the hypnotic pill (unconditioned stimulus) with the routine of taking a pill from the pill bottle (conditioned stimulus).

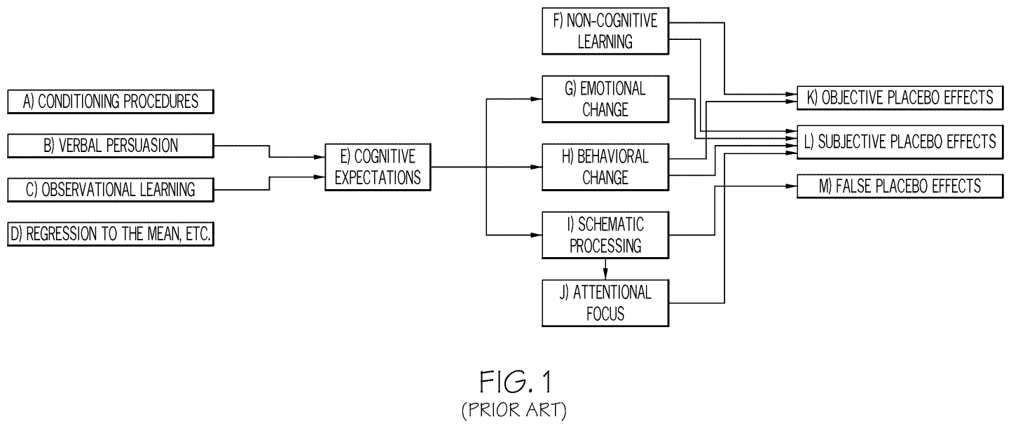

In various aspects, the pairing of the two stimuli may result in improvements in function from both the unconditioned stimulus and conditioned stimulus. Referring to FIG. 1, conditioning (a), in turn, may shape (e) cognitive expectancies, such as the expectancy that the drug will produce better results. Disrupting this pairing could reduce placebo effects through changes in non-cognitive learning and through altered expectancies. An example of (b) verbal persuasion in the context of drugs is direct-to-consumer advertising. Advertisers create (e) positive expectancies for drugs by creating messages about the positive effects of drugs on physiological results. In contrast, therapy and exercises that draw attention to side effects associated with the drug can create (e) negative expectancies for hypnotics. (c) Observational learning may also result in (e) positive expectancies for the drugs. (m) False placebo effects may result from observer bias, which in themselves change (e) cognitive expectancies. As such, (e) cognitive expectancies are important factors in placebo effects and are the ideal target for intervention. In various aspects, such an intervention can use (a) conditioning, (b) verbal persuasion, and (c) observational learning.

As cognitive expectancies in the context of the placebo effect are shaped by several factors and may be modifiable. Embodiments targeting cognitive expectancies, therefore, should produce measurable changes in beliefs and attitudes about drug use. Similarly, nocebo effects may result from embodiments targeting cognitive expectancies, if the goal is to perceive negative effects of a particular treatment.

According to one aspect, this disclosure relates to methods of tapering and/or gradually discontinuing a drug using a blinded or masked approach to reduce placebo and/or nocebo effects that may impede a subject's successful tapering or discontinuation of the drug. In some cases, maximizing a nocebo effect may have therapeutic benefits and itself be a goal. In certain aspects, a blinded or masked approach conceals from the subject the exact daily dose of the drug during the tapering period. In one aspect, over the course of the taper, an increasing amount of inert edible filler can be mixed with the drug. A subject can agree to the plan for tapering with the goal of drug discontinuation and to forego knowledge of the daily dose that is ingested. In various aspects, this blinding or masking can reduce the anxiety and anticipatory unwanted symptoms that can occur when a drug is withdrawn.

In one aspect, disclosed is a method of tapering or uptitrating a drug comprising: receiving, at a computing device, a patient-derived parameter comprising at least one of: a Likert-like scale response indicative of the patient's motivation to begin or continue a drug tapering or uptitrating plan, a Likert-like scale response indicative of the patient's subjective perception of a drug tapering or uptitrating plan, the patient's blood pressure, the patient's heart rate, a Likert-like scale response indicative of the patient's subjective report of global well-being, or Clinical Institute Withdrawal Assessment Scale (CIWA-B) scores for benzodiazepines or non-benzodiazepine sedative hypnotics; receiving, at a computing device, a provider-derived parameter comprising at least one of: a baseline dose, a desired end dose, a number of days per week the drug is taken, a day of the week the drug is taken, a number of weeks for tapering, a number of digits following a decimal place for the drug dose, a day for being drug-free days, a function of the taper plan, a strength of the taper plan, a specific dose strength on certain days, an upper bound for percentage reduction during each week or partial week, a lower bound for percentage reduction during each week or partial week, or a maximal percent reduction per week; generating, by the computing device, based on the at least one patient-derived parameter, the at least one provider-derived parameter, and the drug, a taper or uptitrating plan associated with the drug and the patient, wherein the taper or uptitrating plan is indicative of a plurality of dosage amounts of the drug for each of a plurality of specific days; determining, by the computing device, based on the taper or uptitrating plan, a first dosage amount of the plurality of dosage amounts of the drug; and sending, by the computing device, an indication of the first dosage amount.

In one aspect, the patient derived parameter comprises a Likert-like scale response indicative of the patient's motivation to begin or continue a drug tapering or uptitrating plan; a Likert-like scale response indicative of the patient's subjective perception of a drug tapering or uptitrating plan; the patient's blood pressure; the patient's heart rate; a Likert-like scale response indicative of the patient's subjective report of global well-being; and Clinical Institute Withdrawal Assessment Scale (CIWA-B) scores for benzodiazepines or non-benzodiazepine sedative hypnotics.

In a further aspect, the provider-derived parameter comprises a baseline dose, a desired end dose, a number of days per week the drug is taken, a day of the week the drug is taken, a number of weeks for tapering, a number of digits following a decimal place for the drug dose, a day for being drug-free days, a function of the taper plan, a strength of the taper plan, a specific dose strength on certain days, an upper bound for percentage reduction during each week or partial week, a lower bound for percentage reduction during each week or partial week, and a maximal percent reduction per week.

In a further aspect, the method further comprising receiving, at a computing device, the patient's feedback to the taper or uptitrating plan, and optionally modifying the plan based upon the patient's feedback.

In a further aspect, the indication of the first dosage amount comprises an obscured dosage amount.

In a further aspect, disclosed is a method of tapering or uptitrating a drug comprising: receiving, at a computing device, at least one parameter associated with a drug and a patient identifier; generating, by the computing device, based on the at least one parameter and the drug, a taper plan associated with the drug and the patient identifier, wherein the taper plan is indicative of a plurality of dosage amounts of the drug for each of a plurality of specific days; determining, by the computing device, based on the taper plan, a first dosage amount of the plurality of dosage amounts of the drug; and sending, by the computing device, an indication of the first dosage amount.

According to one aspect, at least one parameter comprises at least one of: a baseline dose, a desired end dose, a number of days per week the drug is taken, a day of the week the drug is taken, a number of weeks for tapering, a number of digits following a decimal place for the drug dose, an upper bound for percentage reduction during each week or partial week, a lower bound for percentage reduction during each week or partial week, or a maximal percent reduction per week.

According to a further aspect, the at least one parameter comprises at least one of: a day for being drug-free days, a function of the taper plan, a strength of the taper plan, or a specific dose strength on certain days.

In a still further aspect, the indication of the first dosage amount comprises an obscured dosage amount.

In one aspect, the method can utilize a number of parameters to determine a dosing schedule and dose for each dosage of the drug. Such parameters include without limitation a baseline (or current) dose, e.g., 10 mg; a desired end dose, e.g., 0 mg; a number of days per week the drug is taken, e.g., 5 days per week or 7 days per week; the days of the week the drug is taken, e.g., Monday-Friday or Sunday-Saturday; a number of weeks for tapering, e.g., 10 weeks; a number of digits following the decimal place for the drug dose (which limits the possible drug doses for drug preparation purposes), e.g., 2 digits, if an allowable drug dose in the taper schedule is 1.25 milligrams; upper and lower bounds for percentage reduction during each week or partial week, e.g., week 1 upper bound=100% of the baseline dose and lower bound=75% of the baseline dose and week 2 upper bound=75% of the baseline dose and lower bound=50% of the baseline dose, or upper and lower bounds for 3 days of week 1 and upper and lower bounds for the other 4 days of week 1; maximal percent reduction per week, e.g., 25% reduction from week 1 to week 2, week 2 to week 3; days for being drug-free days, e.g., always make Saturdays and Sundays drug-free days, even though baseline days of the week entered in prior field above included Saturdays and Sundays; function or “shape” of taper, e.g., linear or inverse variation; strengths of taper, e.g., a dose equivalent of ¾ of the baseline drug dose, ½ of the baseline drug dose, ¼ of the baseline drug dose; and/or specific dose strengths on certain days; or a combination thereof.

According to one aspect, within the above parameters, dosages can vary randomly from day-to-day during each week. In one aspect, for example, the method can generate 7 random doses within the parameters selected and utilized from the above list for each week of the taper. The random doses can be provided to a subject with instructions for which day to take each dosage (e.g., day 1, day 2, etc.). According to a further aspect, the method allows for updating and/or augmenting the parameters during a taper, such as augmenting the function of the taper, increasing the number of weeks of the taper, and/or modification of any other parameter.

According to one aspect, the method can be embodied as software that automatically calculates doses based on the parameters, or a computing device that executes such software. For example, in some aspects, the software can calculate daily dosages based on one or more inputted parameters. In certain aspects, a series of doses (e.g., 7 doses for a week) can be selected at random from values that fall within the inputted upper and lower bounds for that week (e.g., by using a random number generator, assigning the random numbers to each value that falls within the upper and lower bounds, rank ordering the random numbers, and then selecting the values assigned to the lowest random numbers), while maintaining the total weekly dose within the maximum acceptable week-to-week percent reduction parameter.

In some aspects, the doses (e.g., the 7 doses for a week) can be randomly assigned to each day of that week, thereby resulting in a tapering schedule that is not entirely predictable to the subject who is tapering the drug. In various aspects, the upper and lower bound values and the acceptable percent reduction result in a computed tapering plan that will be acceptable to the prescribing healthcare professional. In various aspects, the taper schedule can be saved in electronic or print format and labeled with dose per day (e.g., day 1, day 2, etc.).

According to one aspect, the initial taper plan can be altered by a user after generation of the initial taper plan. For example, certain values can be “locked” (e.g., a specific dose on a specific day or to mark days that have been accomplished) and/or a new set of values can be augmented, such to allow a subject to “plateau” the taper temporarily, the number of weeks of the taper can be adjusted (e.g., to extend the taper). By adjusting certain parameter values, in various aspects, the method can generate a new tapering plan according to the new parameters.

It should be noted that a taper can be in either direction (e.g., taper-up or taper-down), such that the described methods can be used to withdraw from a drug or to titer a drug up to an effective dose. Furthermore, when tapering-down, the overall objective may not be to taper to a zero dose, and a number of aspects of the method can generate a taper plan to a lower dose. Additionally, in certain aspects, the method can involve adjusting the dose based on limitations of splitting or cutting certain pills. For example, if a random dose is calculated to be 1.37 mg, but that particular drug can only be split into 1.5 mg or 1.25 mg doses (because pills only come in 2.5 mg or 3 mg doses), an adjustment of the doses can be accomplished to compensate for these off-split doses.

In various aspects, once a taper plan is developed for a subject, the doses can be administered to the patient in accordance with a treatment plan. In some aspects, doses can be provided to the subject in a masked and/or blinded manner such that the exact concentration of active ingredient in each dose is hidden from the patient. In some aspects, a dose in the plan will be equivalent to a readily available size (e.g., 5 mg, 10 mg, etc.), while certain doses may not be available as a readily available dose (e.g., 2.5 mg, 1.25 mg) and require cutting, splitting, or some other method of generating a smaller or larger dose size.

Thus, in some aspects, the method can utilize a compounding pharmacist, pill splitter, or another method to divide pills into appropriate sizes. In some aspects, doses can be placed into a blinding capsule (e.g., to hide the size of the dose from the user). Blinding capsules are empty pill capsules that allow them to be filled and closed, while hiding the contents from an individual taking the dosage. For example, the capsule can be opaque as to mask the contents held within the capsule. Other aspects can utilize colored capsules, such that the capsule mimics or matches the typical color of the drug being taken by the subject. By providing masked and/or blinded capsules to the subject, it can prevent the subject from identifying the specific dose being taken, thus limiting the effect of the subject knowing that the dose is lower. Maintaining a constant color and/or shape can also maintain the subject's expectations and rituals of taking the particular drug, thus leveraging psychological factors to increase the success of a tapering plan. Conversely, colors associated with a negative connotation for the user may be utilized to engender negative expectancies pertaining to a drug, if desired.

In some aspects, fillers or other ingredients can be used to prevent the dose from being moveable within a capsule or to mask the specific dose. In some aspects, the dose can be ground into a powder and mixed with a filler to further obfuscate or mask the dose from the subject taking the dose. Several appropriate fillers are known in the art for pharmaceutical formulations and selection of a suitable filler will be appreciated by one of ordinary skill in the art. In some aspects, fillers and capsules that provide similar benefits or characteristics or do not interfere with the delivery of the initial drug can be used. For example, if the initial drug has a time release formulation, the blinded and/or masked version can possess a similar release profile, or can maintain the same release profile (e.g., the masked and/or blinded version does not increase or decrease the release time of the drug).

In some aspects, when individual doses are being created, the capsules may differ from the shape or color of an initial drug. As such, certain embodiments will delay an actual taper for a time to condition the individual to the new color, shape, and/or other characteristics of the new dosages.

In some aspects, the dosages can be provided in daily or weekly specific doses. For example, some embodiments can provide a series of doses labeled or marked with specific days (e.g., day 1, day 2, etc.) for a portion or all of the taper term. In some aspects, the dosages can be provided in blister packs or some other means of providing individual doses. Certain embodiments can provide doses on a weekly basis, where the pills within the week can be taken in any order, such to assist in randomizing the doses during that time period.

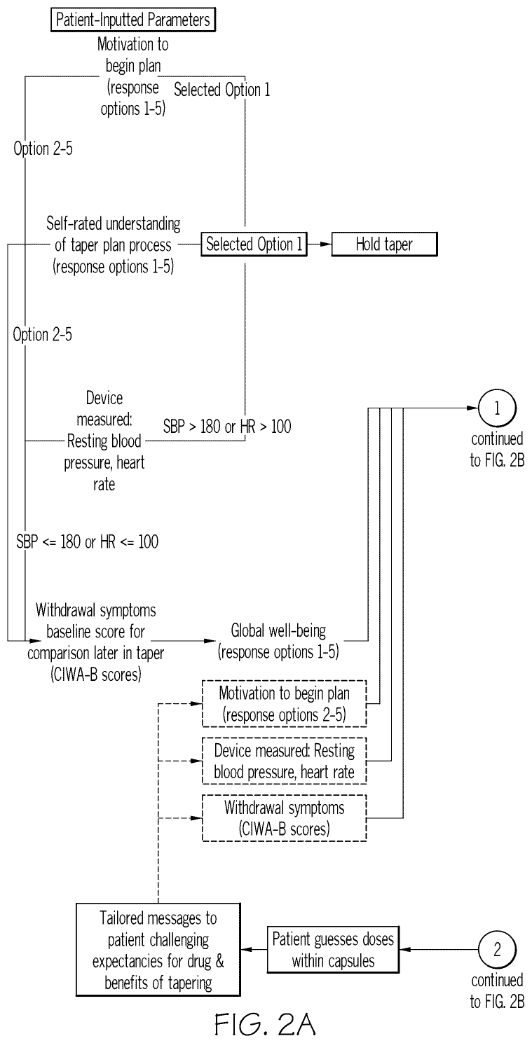

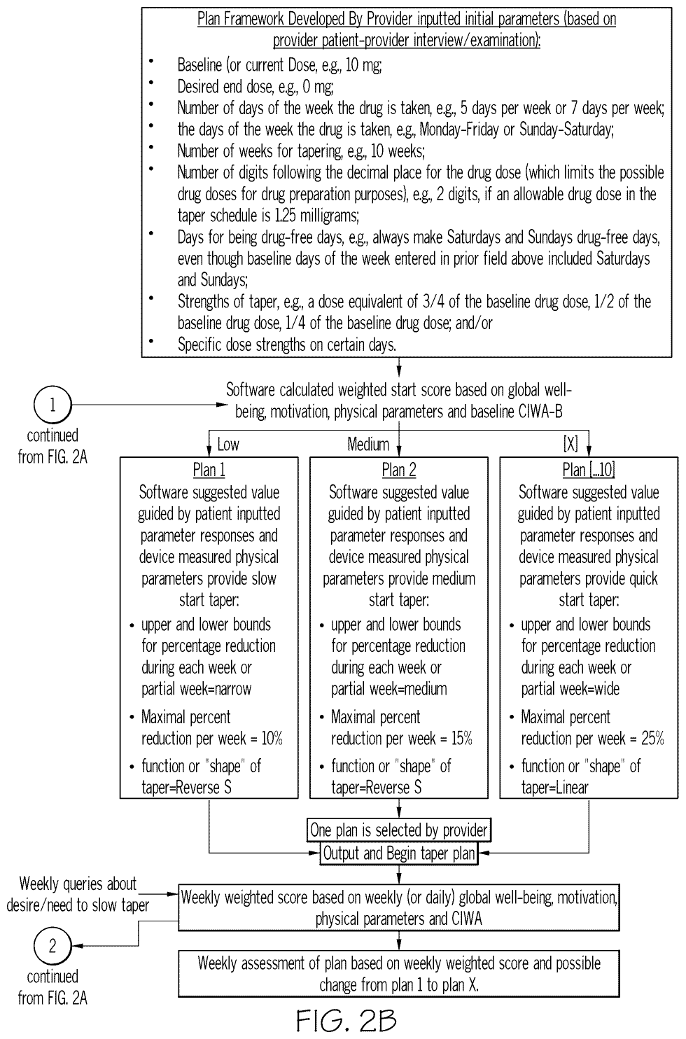

FIG. 2 is a flow-chart illustrating an exemplary algorithm that can be used to implement the described tapering methods. In one aspect, the subject enrolled in the drug tapering program or a healthcare professional assisting the subject can input parameters via software or an application on a computing device (e.g., a desktop device, tablet, handheld device, and the like). In one aspect, the subject or a healthcare professional working with the subject can input data into the following fields: (1) motivation to begin or continue plan, measured on a Likert-like scale; (2) subjective perception of understanding of the taper plan process, measured on a Likert-like scale; (3) transmission of real-time blood pressure and heart-rate (HR) reading accessed through included apparatus; (4) subjective report of global well-being, measured on a Likert-like scale; and (5) Clinical Institute Withdrawal Assessment Scale (CIWA-B) scores for those subjects tapering off benzodiazepines or non-benzodiazepine sedative hypnotics, with CIWA-B queries delivered directly from the software. In a further aspect, (6) on-screen reminder of the benefits of tapering/discontinuation/uptitration; and (7) query to patient regarding desire to slow the rate of the taper or to continue at current pace.

In one aspect, the medication prescriber can utilize data interpreted from the above inputs to inform speed of the taper method. For example, a response to (2) above can reveal a lack of understanding of how the taper plan works and can result in the prescriber slowing the taper plan to allow for further education about the plan. Based on the above inputs, in one aspect, software can generate a recommendation to the health care provider regarding pace of taper and recommendations addressing “trouble points” that may be reflected by objective and/or subjective patient data received.

In a further aspect, the software used to implement the method can also provide feedback about the taper to the patient that is designed to increase positive expectations regarding the success of the taper and increase negative expectations surrounding the use of medication in a taper plan, or reduce negative expectations in a medication uptitration plan. Feedback messages may be based on 1) mathematical difference (positive, zero, or negative value) between the patient-inputted guesses about the patient's most recent doses in the capsule and the actual doses contained in the capsules in combination with 2) patient-inputted well-being scores and for example, the Clinical Institute Withdrawal Assessment Scale (CIWA-B) scores for those subjects tapering off benzodiazepines or non-benzodiazepine sedative hypnotics. The algorithm of the messages will not be delivered in a manner that would enable the patient to unblind during the taper (random neutral messages will be interspersed in the delivery of messages). These messages are the main component of expectancy modules.

Referring to FIG. 2, an exemplary method can comprise the prescriber and patient meeting to discuss the taper or uptitration plan. The patient or a healthcare professional assisting the patient can input the patient-inputted parameters into a computing device. These parameters can include: (1) motivation to begin or continue plan, measured on a Likert-like scale; (2) subjective perception of understanding of the taper plan process, measured on a Likert-like scale; (3) transmission of real-time blood pressure and heart-rate (HR) reading accessed through included apparatus; (4) subjective report of global well-being, measured on a Likert-like scale; and (5) Clinical Institute Withdrawal Assessment Scale (CIWA-B) scores for those subjects tapering off benzodiazepines or non-benzodiazepine sedative hypnotics, with CIWA-B queries delivered directly from the software; or any combination thereof.

In some aspects, a plan framework can be developed by the healthcare provider based upon the patient-inputted parameters and other variables discussed at the meeting. The plan can be developed based on the following parameters: Baseline (or current) dose, e.g., 10 mg; Desired end dose, e.g., 0 mg; Number of days per week the drug is taken, e.g., 5 days per week or 7 days per week; the days of the week the drug is taken, e.g., Monday-Friday or Sunday-Saturday; Number of weeks for tapering, e.g., 10 weeks; Number of digits following the decimal place for the drug dose (which limits the possible drug doses for drug preparation purposes), e.g., 2 digits, if an allowable drug dose in the taper schedule is 1.25 milligrams; Days for being drug-free days, e.g., always make Saturdays and Sundays drug-free days, even though baseline days of the week entered in prior field above included Saturdays and Sundays; Strengths of taper, e.g., a dose equivalent of ¾ of the baseline drug dose, ½ of the baseline drug dose, ¼ of the baseline drug dose; and/or Specific dose strengths on certain days; or a combination thereof.

Based on a combination of patient-inputted parameters and the plan framework, the software can calculate a weighted start score based on global well-being, motivation, physical parameters, and in the case of benzodiazepines, CIWA-B scores. Based on the weighted score, for example, a variety of plans can be devised. For example, low-taper plan can be devised that includes upper and lower bounds for percentage reduction during each week or partial week, which can be a narrow maximal percent reduction per week, e.g., 10%, as a function of shape of the taper, such as a Reverse S curve. A medium plan can be devised with upper and lower bounds for percentage reduction during each week or partial week, e.g., medium maximal percent reduction per week, e.g., 15% function of shape of the taper, e.g., Reverse S curve. In some aspects, the taper or uptitration plan can be modified based upon ongoing patient and healthcare provider feedback.

Once a taper or uptitration plan begins, the software can generate weekly weighted scores based on weekly (or daily) global well-being, motivation, physical parameters, and in the case of benzodiazepine, CIWA scores. From these scores, there can be a weekly assessment of the plan based on weekly weighted scores, which can result in a change or modification to the taper or uptitration plan. The weekly weighted scores can also be used to generate tailored messages to the patient challenging expectancies for drug benefits and tapering as part of the expectancy modules. This can be done in conjunction with the patient guessing the amount of the drug in the blinded or masked dosage form. In addition, there can be weekly queries about the desire or need to slow or speed up the taper.

In some aspects, when dosing medication to the patient, upper and lower bounds can be delivered using randomization process to a medication provider apparatus, such as the one described below, with the delivery of the medication to the patient on a particular plan. Following dosing of medication, the patient can input user-generated responses to a computing device, with data being sent to the prescriber, who may alter dosing or speed of the plan based upon the results. Plan dosing or speed may also not change based upon this data. Expectancy modules embedded in the software can promote cognitive shifts in that patient, and these shifts can promote an efficient taper or uptitration through the process.

### C. Apparatus and Methods of Use

In one aspect, disclosed is an apparatus for generating specific masked or blinded doses. The disclosed apparatus can be used in conjunction with a disclosed method. In some aspects, the pill cutter can generate doses in accordance with a taper or uptitrating plan and distribute the doses as a random set for a specific time frame (e.g., 7 pills for week 1), or the pills can be individually delivered as day 1, day 2, etc. for the taper plan.

In one aspect, a disclosed pill cutting or splitting apparatus can comprise a handle to allow for actuation of a cutting mechanism, such as a blade, wedge, or other apparatus for cutting or splitting an object. Certain aspects can include a returning mechanism, such as a spring, to return the handle to a starting position and allow for the next actuation of the blade using the handle. Further aspects can include a platform for holding a pill or drug in place. In certain aspects, the platform includes an indentation or other texture to maintain a specific placement or orientation of the pill. Additional aspects include ridges, or raised edges to prevent pill pieces from being lost and/or falling off the platform.

In some aspects, the apparatus can include a lower portion or a torus or disk. In a number of aspects, the disk can be divided along a lateral axis to allow separation of a top half and a bottom portion. The lower portion of the disk can include a number of cavities to hold capsules provided by a user, thus allowing for easier encapsulation of pill fragments into the capsules. In a number of aspects, the top, handle portion can be secured to the disk with hooks or another method for securing two portions. In some aspects, the handle portion can be removed and inserted into the disk.

In some aspects, once capsules placed in the cavities are filled with pill fragments and/or fillers, the top off portion of the disk can be removed to allow for the capsules to be sealed and/or covered. The top portion of the disk can be replaced, which allows for shaking or inverting the disk to allow for collection of the capsules.

It should also be noted that a pill cutter can be automated, such that the individual capsules can be automatically filled and/or produced in accordance with a taper or uptitrating plan without the use of a human to press a handle and/or actuate the cutting mechanism.

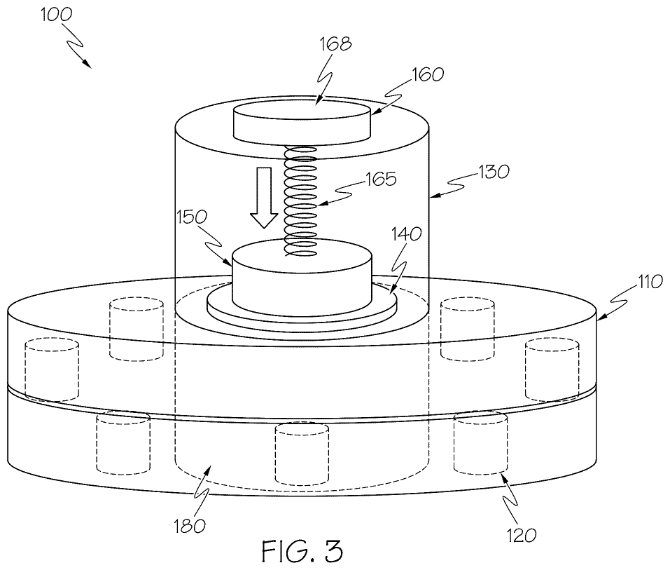

With reference to FIG. 3, an exemplary apparatus 100 can comprise a body 110 having a central axis. Body 110 can define a plurality of receptacles 120 that are spaced around the central axis. Receptacles 120 can define a perimeter. Pill cutter 130 can be disposed on at least a portion (e.g., a segment) of the perimeter defined by receptacles 120.

In some aspects, pill cutter 130 can comprise platform 140, which can be configured to receive a pill. Pill cutter 130 can also comprise cutting element 150. The cutting element can be a blade, wedge, and the like. Cutting element 150 can be coupled to platform 140 and configured to move toward and away from platform 140 in order to cut the pill received on platform 140.

Pill cutter 130 can further comprise actuator 160. Actuator 160, in some aspects, can comprise a biasing element, e.g., spring 165, that is configured to bias the blade away from platform 140. In a further aspect, actuator 160 can comprise a handle, e.g., handle 168, that can be coupled to the cutting element and configured to receive a force in a direction toward platform 140 to drive the cutting mechanism toward the platform.

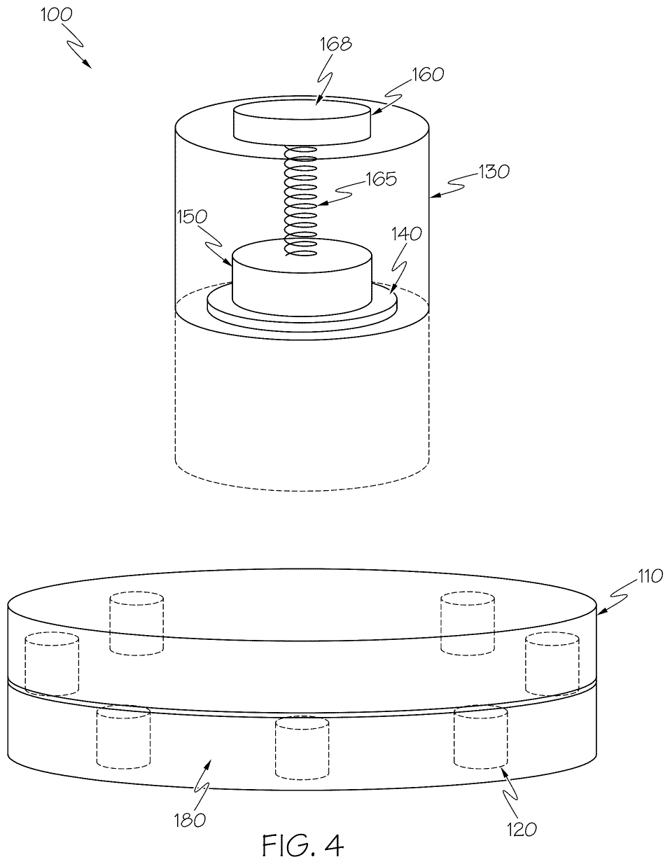

In one aspect, body 110 can define bore 180. According to this aspect, pill cutter 130 can comprise a support that can be selectively positionable within bore 180 of body 110. Thus, in some aspects, with reference to FIG. 4, the pill cutter can be removable from the bore of the central body.

According to one aspect, pill cutter 130 can be rotatable relative to body 110 about a central axis of the body. Thus, for example, pill cutter 130 can be positioned and selectively moved around the perimeter defined by receptacles 120 such that a capsule in each receptacle can be filled with a pill fragment made with the pill cutter. Alternatively, pill cutter 130 can remain fixed, and body 110 can be rotated such that each rotation results in a different segment of the perimeter defined by the receptacles to be underneath the pill cutter.

According to one aspect, receptacles 120 can be configured to receive at least a portion of a respective capsule, e.g., a capsule capable of receiving a drug or medication. Such capsules are known in the art.

In a further aspect, body 110 comprises an upper portion that can be separable from the lower portion. The lower portion can define receptacles 120, while the upper portion can be configured to cover the plurality of receptacles 120.

In some aspects, platform 140 can define at least one pill retaining feature. The pill retaining feature can comprise an indentation, texture, or similar feature. Platform 140 can also comprise in some aspects a mechanism to push the pill fragment off the platform into an empty capsule. Alternatively, in some aspects, a user can manually push the pill fragment into the capsule.

In a further aspect, disclosed is a method of using the described apparatus, the method comprising: placing a pill on the platform; cutting the pill into at least two fragments; and moving at least one fragment of the at least two fragments into a first receptacle of the plurality of receptacles and at least one other fragment of the at least two fragments into a second receptacle.

In one aspect, the method of using the apparatus is suitable for blinding or masking the dosage in each capsule. Thus, for example, in some aspects, the first receptacle is not adjacent to the second receptacle. In a further aspect, the first and second receptacle are on opposing sides of the pill cutter.

In one aspect, the method further comprises rotating the pill cutter relative to the body; cutting a second pill into at least two fragments; and moving at least one fragment of the at least two fragments of the second pill into a third receptacle of the plurality of receptacles and at least one other fragment of the at least two fragments of the second pill into a fourth receptacle.

In a further aspect, the method comprises: positioning at least a portion of a capsule within each of the first receptacle and the second receptacle before moving the at least one fragment of the at least two fragments into the first receptacle of the plurality of receptacles and the at least one other fragment of the at least two fragments into the second receptacle; and sealing the at least a portion of the capsule positioned within each of the first receptacle and the second receptacle.

It will be apparent to those skilled in the art that various modifications and variations can be made without departing from the scope or spirit of this disclosure. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A method of tapering or uptitrating a drug for a patient, comprising:
receiving, at a computing device, a patient-derived parameter comprising at least one of: a Likert-like scale response indicative of the patient's motivation to begin or continue a drug tapering or uptitrating plan, a Likert-like scale response indicative of the patient's subjective perception of a drug tapering or uptitrating plan, the patient's blood pressure, the patient's heart rate, a Likert-like scale response indicative of the patient's subjective report of global well-being, or Clinical Institute Withdrawal Assessment Scale (CIWA-B) scores for a benzodiazepine or non-benzodiazepine sedative hypnotic;
receiving, at a computing device, a provider-derived parameter comprising at least one of: a baseline dose, a desired end dose, a number of days per week the drug is taken, a day of the week the drug is taken, a number of weeks for tapering, a number of digits following a decimal place for the drug dose, a day for being drug-free days, a function of the taper plan, a strength of the taper plan, a specific dose strength on certain days, an upper bound for percentage reduction during each week or partial week, a lower bound for percentage reduction during each week or partial week, or a maximal percent reduction per week;
generating, by the computing device, based on the at least one patient-derived parameter, the at least one provider-derived parameter, and the drug, a taper or uptitrating plan associated with the drug and the patient, wherein the taper or uptitrating plan is indicative of a plurality of dosage amounts of the drug for each of a plurality of specific days;
determining, by the computing device, based on the taper or uptitrating plan, a first dosage amount of the plurality of dosage amounts of the drug; and
sending, by the computing device, an indication of the first dosage amount.

2. The method of claim 1, wherein the patient derived parameter comprises a Likert-like scale response indicative of the patient's motivation to begin or continue a drug tapering or uptitrating plan; a Likert-like scale response indicative of the patient's subjective perception of a drug tapering or uptitrating plan; the patient's blood pressure; the patient's heart rate; a Likert-like scale response indicative of the patient's subjective report of global well-being; and Clinical Institute Withdrawal Assessment Scale (CIWA-B) scores for benzodiazepines or non-benzodiazepine sedative hypnotics.

3. The method of claim 1, wherein the provider-derived parameter comprises a baseline dose, a desired end dose, a number of days per week the drug is taken, a day of the week the drug is taken, a number of weeks for tapering, a number of digits following a decimal place for the drug dose, a day for being drug-free days, a function of the taper plan, a strength of the taper plan, a specific dose strength on certain days, an upper bound for percentage reduction during each week or partial week, a lower bound for percentage reduction during each week or partial week, and a maximal percent reduction per week.

4. The method of claim 1, further comprising receiving, at a computing device, the patient's feedback to the taper or uptitrating plan, and optionally modifying the plan based upon the patient's feedback.

5. The method of claim 1, wherein the indication of the first dosage amount comprises an obscured dosage amount.

6. A method of tapering or uptitrating a drug comprising:
receiving, at a computing device, at least one parameter associated with a drug and a patient identifier;
generating, by the computing device, based on the at least one parameter and the drug, a taper plan associated with the drug and the patient identifier, wherein the taper plan is indicative of a plurality of dosage amounts of the drug for each of a plurality of specific days;
determining, by the computing device, based on the taper plan, a first dosage amount of the plurality of dosage amounts of the drug; and
sending, by the computing device, an indication of the first dosage amount.

7. The method of claim 6, wherein the at least one parameter comprises at least one of: a baseline dose, a desired end dose, a number of days per week the drug is taken, a day of the week the drug is taken, a number of weeks for tapering, a number of digits following a decimal place for the drug dose, an upper bound for percentage reduction during each week or partial week, a lower bound for percentage reduction during each week or partial week, or a maximal percent reduction per week.

8. The method of claim 6, wherein the at least one parameter comprises at least one of: a day for being drug-free days, a function of the taper plan, a strength of the taper plan, or a specific dose strength on certain days.

9. The method of claim 6, wherein the indication of the first dosage amount comprises an obscured dosage amount.

10. An apparatus comprising:
a body having a central axis, wherein the body defines a plurality of receptacles that are spaced around the central axis, wherein the plurality of receptacles define a perimeter;
a pill cutter disposed on a segment of the perimeter defined by the plurality of receptacles, the pill cutter comprising:
a platform that is configured to receive a pill;
a cutting element that is coupled to the platform and configured to move toward and away from the platform in order to cut a pill received on the platform; and
an actuator that is configured to cause the cutting element to move toward and away from the platform.

11. The apparatus of claim 10, wherein the actuator comprises:
a biasing element that is configured to bias the cutting element away from the platform; and
a handle that is coupled to the cutting element and configured to receive a force in a direction toward the platform to drive the cutting element toward the platform.

12. The apparatus of claim 11, wherein the biasing element is a spring.

13. The apparatus of claim 10, wherein the body defines a bore, wherein the pill cutter comprises a support that is selectively positionable within the central bore of the body.

14. The apparatus of claim 10, wherein the pill cutter is rotatable relative to the body about the central axis.

15. The apparatus of claim 10, wherein the receptacles are each configured to receive at least a portion of a respective capsule.

16. The apparatus of claim 10, wherein the body comprises an upper portion that is separable from the lower portion, wherein the lower portion defines the plurality of receptacles, wherein the upper portion is configured to cover the plurality of receptacles.

17. The apparatus of claim 10, wherein the platform defines at least one pill retaining feature.

18. A method of using the apparatus of claim 10, the method comprising:
placing a pill on the platform;
cutting the pill into at least two fragments; and
moving at least one fragment of the at least two fragments into a first receptacle of the plurality of receptacles and at least one other fragment of the at least two fragments into a second receptacle.

19. The method of claim 18, wherein the first receptacle is not adjacent to the second receptacle.

20. The method of claim 19, wherein the first and second receptacle are on opposing sides of the pill cutter.
